(19) United States
(12) Patent Application Publication
(10) Pub. No.: US 2016/0263310 A1
(43) Pub. Date: Sep. 15, 2016

(54) Mobile One-Handed Tube-Feeding Syringe Support

(71) Applicant: Jason B. Helbig, Traverse City, MI (US)

(72) Inventor: Jason B. Helbig, Traverse City, MI (US)

(21) Appl. No.: 15/047,147

(22) Filed: Feb. 18, 2016

Related U.S. Application Data
(60) Provisional application No. 62/132,527, filed on Mar. 13, 2015.

(51) Int. Cl. A61M 5/00; F16M 11/06; F16M 11/24; F16M 11/04

(52) U.S. Cl. CPC A61M 5/008; F16M 11/043; F16M 11/041; F16M 11/06; F16M 11/24; A61J 15/0026

(57) ABSTRACT

A tube feeding syringe holder having a continuously flexible, shape-holding arm supporting a drop-in syringe receiver at its terminal end. The syringe receiver is a generally circular, non-expanding collar with a bore sized to permit a syringe to be dropped into place axially, in a one-handed operation. The apparatus allows the feeding operation to be hands-free once started. In a preferred form the flexible arm is mounted on a tripod base with a vertically adjustable support post.

### RELATED APPLICATIONS/PRIORITY BENEFIT CLAIM

This application claims the benefit of U.S. Provisional Application No. 62/132,527, filed Mar. 13, 2015 by the same inventor (Helbig), the entirety of which provisional application is hereby incorporated by reference.

### FIELD

The subject matter of the present application is in the field of supports for tube-feeding or “enteral” syringes.

### BACKGROUND

Some people with health problems that affect oral feeding or eating are fed a liquid supplement via flexible tube connected temporarily to the stomach. A tube connected to the stomach through the nose or mouth is usually used for short term feeding supplementation. For longer term nutritional needs, a gastric or “G” tube is connected to a surgically installed port communicating with the stomach. The liquid supplement is delivered from a bag by gravity for larger amounts, or from a syringe (either by gravity or with a plunger) for smaller amounts.

Tube feeding involves the handling of several pieces of equipment and the monitoring and delivery of the supplement, requiring both time and attention on the part of the person receiving the supplement and any caregiver assisting with the feeding. It is often frustrating for the tube-feed operator to find a stable, clean place to secure the syringe, particularly for a gravity feed, and especially if the operator needs or wants hands free to do something else during the feeding (read; hold or attend to the person being fed; answer a phone call; etc.).

Some prior attempts to solve the above problems are known, but appear to have achieved little recognition due to some potential limitations.

The Jackson Peg Tube Stand (www.jacksonpegtubestands.com) is designed as a holder tool to support a PEG feeding tube and used to deliver food product or medications to an enteral nutrition patient by anchoring the PEG feeding tube in a syringe holder. The holder device has a vertically adjustable post supported on a flat pedestal base, and is portable. The base, however, appears to require a stable, cleared, flat surface such as a table or shelf, which might not always be at hand. Also, the syringe appears to be held by a horizontal spring clip or clamp that would require two hands and a decent amount of force to secure the syringe to the vertical post prior to feeding.

The Self Tube Feeder is similar to the Jackson Peg Tube Stand, but uses an H-shaped base made from PVC tubing to support the vertical syringe-holding post. The spring clamp for securing the syringe to the post appears to be the same as in the Jackson device.

### BRIEF SUMMARY

The present invention is a tube feeding syringe holder, useful for any type of tube feeding requiring the use of an inverted syringe. The syringe holder comprises a base with three or more adjustable legs (hereafter a “tripod”), a continuously flexible “flex” support arm connected to and extending from the base, and a drop-in axial syringe receiver or “collar” on the end of the flex arm. The base is free-standing and sufficiently stable on both even and mildly uneven surfaces to hold full syringes at virtually any location relative to the tripod base within an arc defined by the length of the flex arm.

The syringe receiver on the end of the flex arm comprises a horizontal rigid collar with a vertical bore, and the barrel of an inverted syringe dropped axially into the bore of the collar is positively stopped and held in the collar by a shoulder or flange on the syringe. Optionally, the collar's vertical bore has a narrowing taper from top to bottom, i.e. an upper entry diameter greater than a lower exit diameter of the collar independent of any external structure on the syringe barrel. It will be understood that the terms “horizontal” and “vertical” as used with respect to the syringe-holding collar are merely convenient terms to distinguish the generally perpendicular relationship of the collar body and bore, and refer to generally preferred orientations during use.

In a further form the flex arm is attached to the tripod base with a pivoting or rotating connection, and/or via a telescoping or height-adjustable post.

In a further form, the rigid syringe-holding collar is a split, rigid (non-expanding) collar with a front portion open for viewing graduated markings on a syringe barrel or the contents of the syringe.

In a further form, the syringe-holding collar is split off-center, with a side or angled portion open to make it easier for a person sitting to the side of the syringe holder to insert a tube-coupled syringe into the collar.

In still a further form, the syringe-holding collar is connected to the flex arm with a rotatably adjustable quick-connect push fitting, simplifying one handed connection and allowing the angle of the collar (and a syringe therein) to be adjusted relative to horizontal. In particular, the off-center split of the collar allows the collar to be rotated to a vertical position for use as a temporary hook for the tube or for a gravity-feeding bag (which holds on average three times the volume of fluid compared to a syringe) when the syringe is removed. The flex arm is strong enough to hold its position and shape under the weight of reasonable loads hanging from the collar, and may also have a rotatably adjustable quick-connect to the tripod.

These and other features and advantages of the invention will become apparent from the detailed description below, in light of the accompanying drawings.

### BRIEF DESCRIPTION OF THE DRAWINGS

FIG. 1 is a perspective view of an exemplary feeding tube syringe holder according to the invention, in use by a caregiver and patient, and with different positions shown in phantom lines.

FIG. 2 is similar to FIG. 1, but with a vertical extension portion of the holder shown retracted in phantom lines.

FIG. 3 is an exploded assembly view of the main parts of the holder of FIG. 1, showing various tripod leg adjustments in solid and phantom lines.

FIG. 4 is a perspective detail view of the syringe-receiving collar portion of the holder of FIG. 1, with a syringe shown partially and fully inserted in solid and phantom lines.

FIG. 5 is a side elevation detail view of the syringe-receiving collar portion of the holder of FIG. 1.

FIG. 6 is a perspective view of a holder similar to FIG. 1, with a modified base.

FIG. 7 is similar to FIG. 4, but shows a modified collar with an off-center split opening and a quick-connect fitting to the flex arm.

FIG. 8 is similar to FIG. 3, but showing the modified collar of FIG. 7 and rotatable quick connect couplings at both ends of the flex arm.

FIG. 9 is an enlarged detail of the modified collar at the end of the flex arm from FIG. 7, with the collar rotated to a vertical position for use as a hook.

FIG. 10 is a side elevation view of a furniture-mounting clamp used instead of a tripod to support the flex arm and collars of FIGS. 1-9.

FIG. 11 is a side elevation of a wall-mounting bracket used instead of a tripod to support the flex arm and collars of FIGS. 1-9.

FIG. 12 is a perspective view of an alternate wall-mounting bracket used in a manner similar to that of FIG. 11.

FIG. 13 is a perspective view of a tray accessory for a tripod having a vertical post like that in FIGS. 1 and 2.

### DETAILED DESCRIPTION

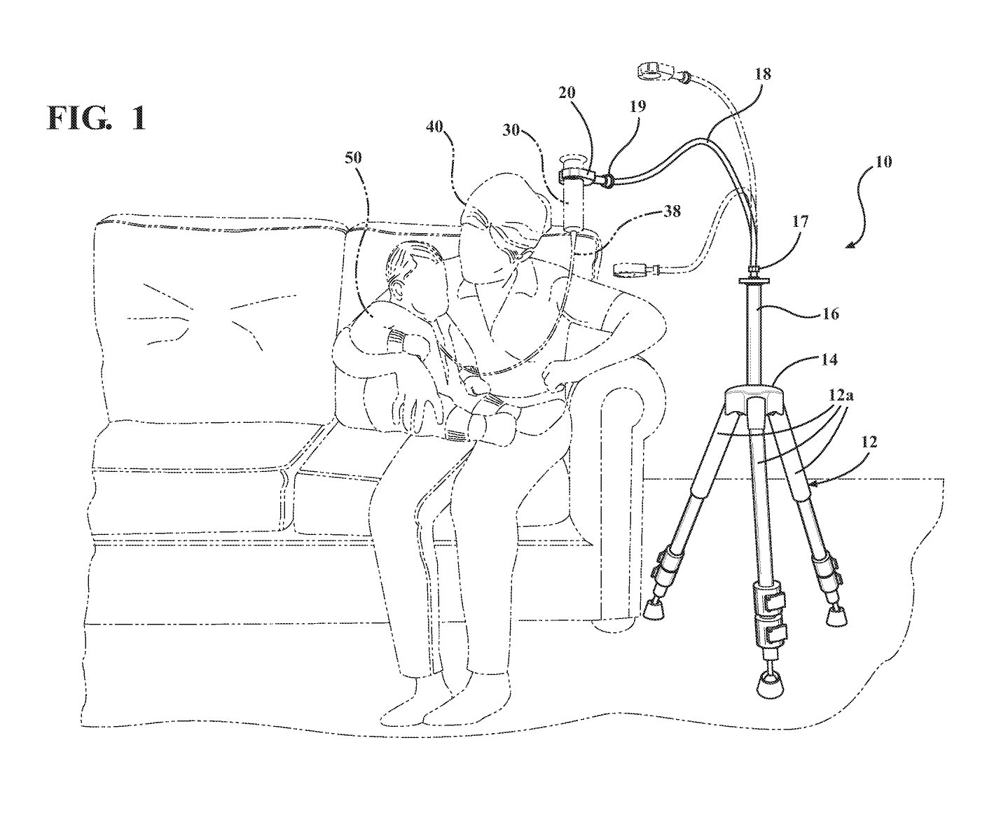

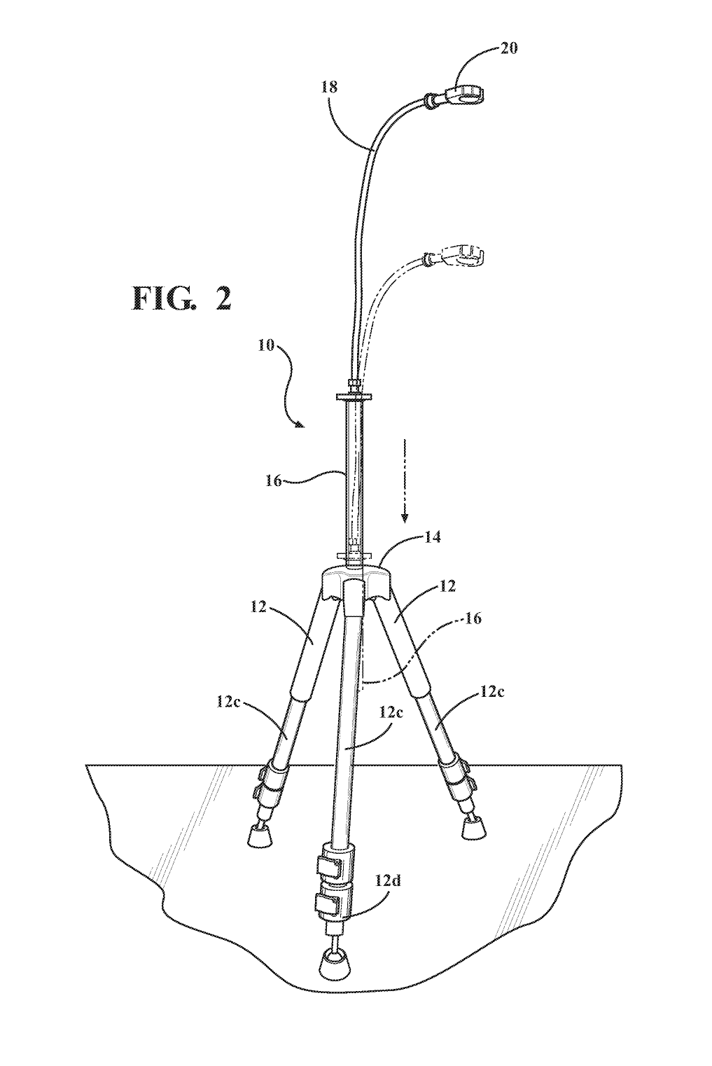

Referring first to FIGS. 1 and 2, a syringe holder 10 according to the invention is shown in exemplary form in order to teach how to make and use the claimed invention. Syringe holder 10 includes a tripod base 12 having three or more legs 12a, a flex arm hub or platform 14 at the apex of legs 12, and a flex arm 18 extending up and out from the platform 14. Flex arm 18 terminates in a syringe receiver or collar 20 adapted to secure a tube feeding syringe 30 in an upside-down, generally vertical position.

In a further and preferred form, flex arm 18 is connected to platform 14 through a height-adjustable boom or post 16, best shown in FIG. 2. Post 16 may be a telescoping multi-piece structure with locked positions at different heights, or it may be a fixed-length post slidingly adjustable up and down through a suitable bore in platform 14, as shown, and secured with a thumbscrew, friction lock, or other known securing device.

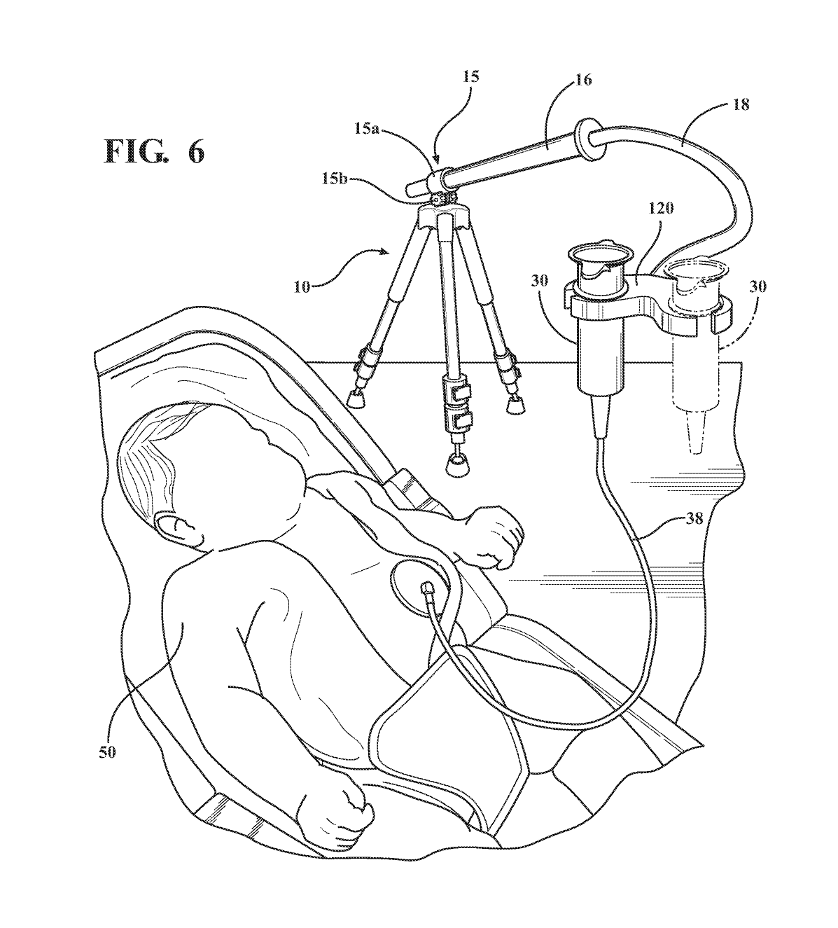

Post 16 may be a fixed-angle post, adjustable only vertically, as in FIGS. 1-5. Alternately, post 16 may be connected to platform 14 with an angle-adjustment mechanism 15 as shown in FIG. 6, for example a pivotally mounted collar 15a adjustably secured at different angles with a thumbscrew type mechanism 15b. Those familiar with tripods used for various purposes, for example photography, will recognize various angle-adjustment mechanisms as being possible. Fixed tripods and tripods with different adjustment options are also possible, and it should be understood that the illustrated example is not intended to be unduly limiting.

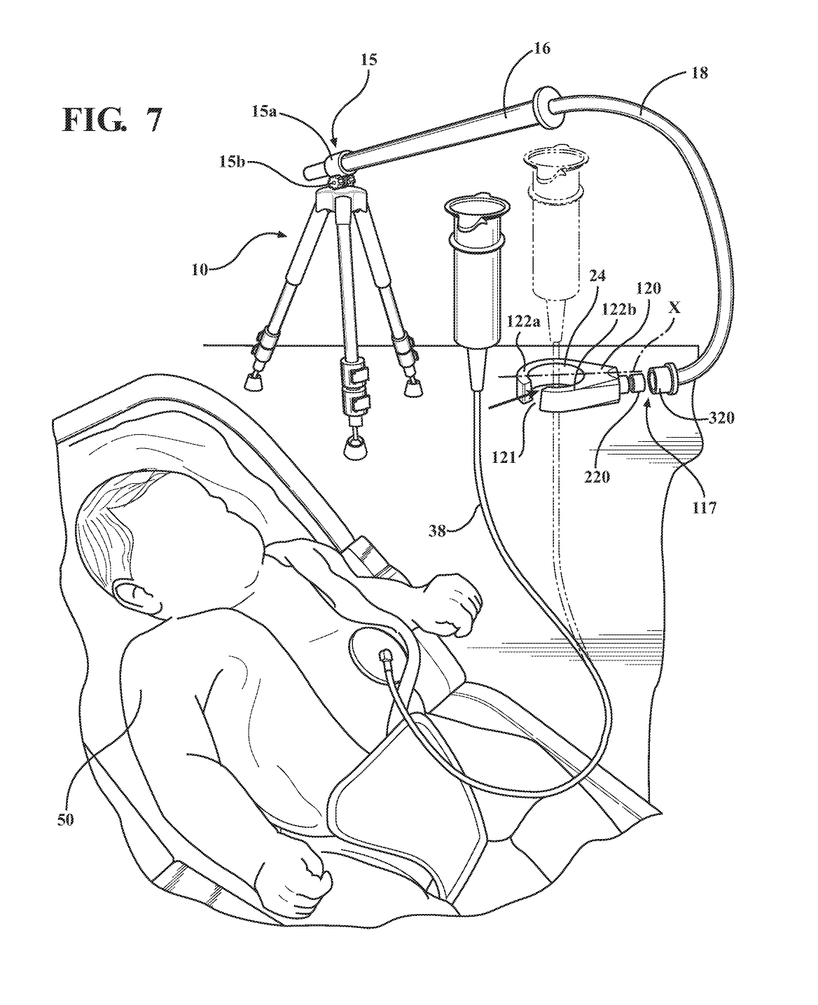

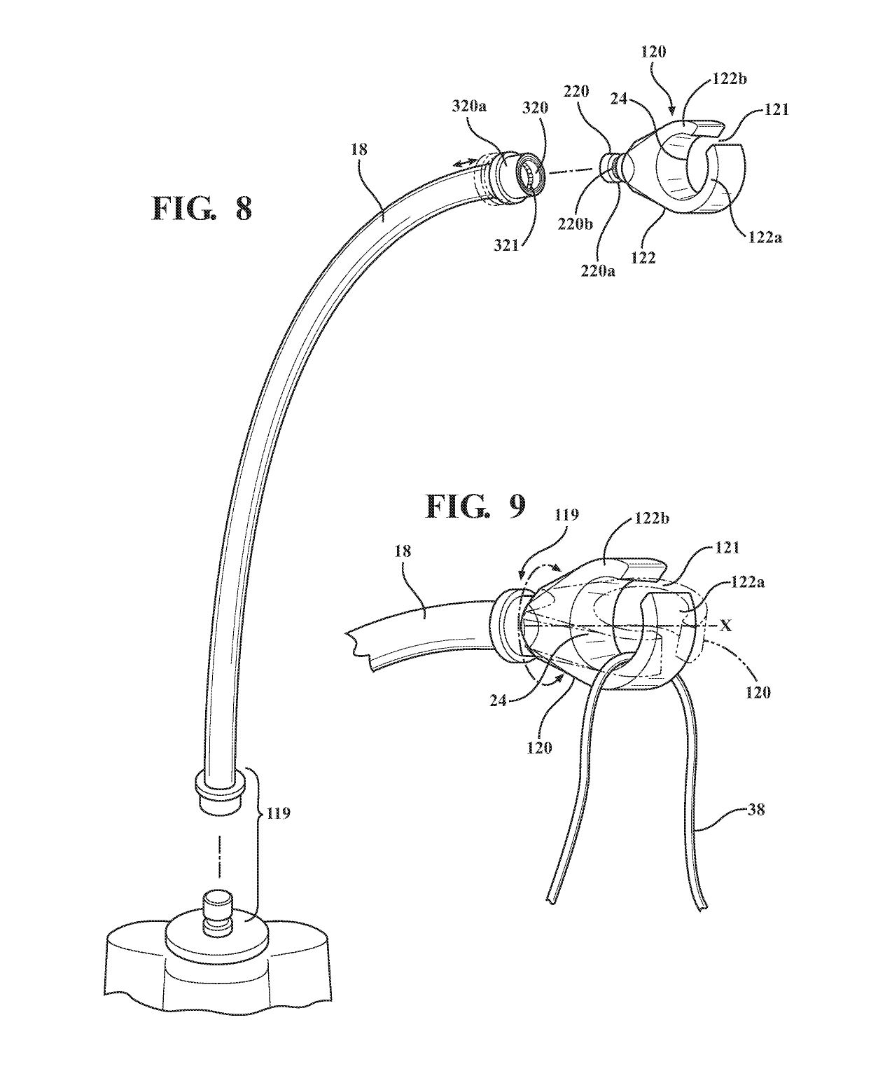

Flex arm 18 in the illustrated example is removably connected at its lower end to an upper end 16a of post 16, for example with a threaded, screw-on connection 17. The connection between flex arm 18 and post 16 is preferably a removable one, although it is also possible to permanently mount the flex arm to the post. The connection 17 between flex arm 18 and post 16 may also be a rotatable connection after the connection is made secure, with suitable rotatable fittings, for example where a snap-on fitting (see FIGS. 7 and 8) is used. It will be understood that a connection platform such as 16a or a connection fitting such as 17 for the lower end of flex arm 18 could be fixed directly on tripod hub 14, rather than on a telescoping post as shown in the illustrated example.

As suggested by the phantom positions in FIGS. 1 and 2, flex arm 18 in the illustrated example is continuously flexible and shape-holding over substantially its entire length except where it is connected to the base platform 14 or post 16, and to the receiver collar 20, and may be for example a flexible gooseneck extension arm of a commercially available type, for example as commonly used in photography for supporting a camera flash at different positions. Flex arm 18 may be bent to a nearly limitless number of shapes, and once bent or curved to a particular shape the arm is self-sustaining in that position, i.e. holds its shape until readjusted, even while holding the weight of a syringe, supplement, and feeding tube. The infinite adjustability of flex arm 18 over its range of motion allows the angle of the syringe to be precisely adjusted to permit precise control over the flow rate of liquid food.

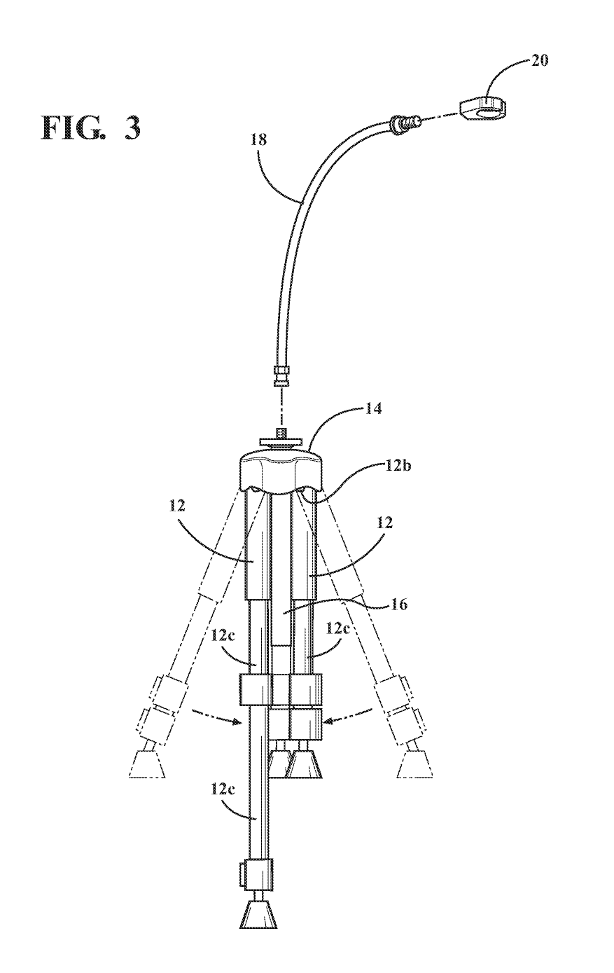

Referring to FIG. 3, tripod legs 12a are preferably pivotally connected at platform or hub 14, so that they can be spread out and secured at different angles from vertical for varying degrees of support, as needed to accommodate various surfaces. The mechanism 12b for rotatably adjusting and securing legs 12a can vary, and in the illustrated example comprises a locking pivot mechanism of a known type, for example as used in photographer's or other types of commercially available tripods.

Tripod legs 12a are preferably also individually adjustable in length, using known types of telescoping leg sections 12c and locking mechanisms 12d to adjust the height of the platform 14 and/or to accommodate mildly uneven support surfaces. Again, the specific mechanism for length adjustment and securing can vary according to known principles, and those familiar with such tripods (for example photographer's tripods) will also be familiar with a variety of suitable mechanisms.

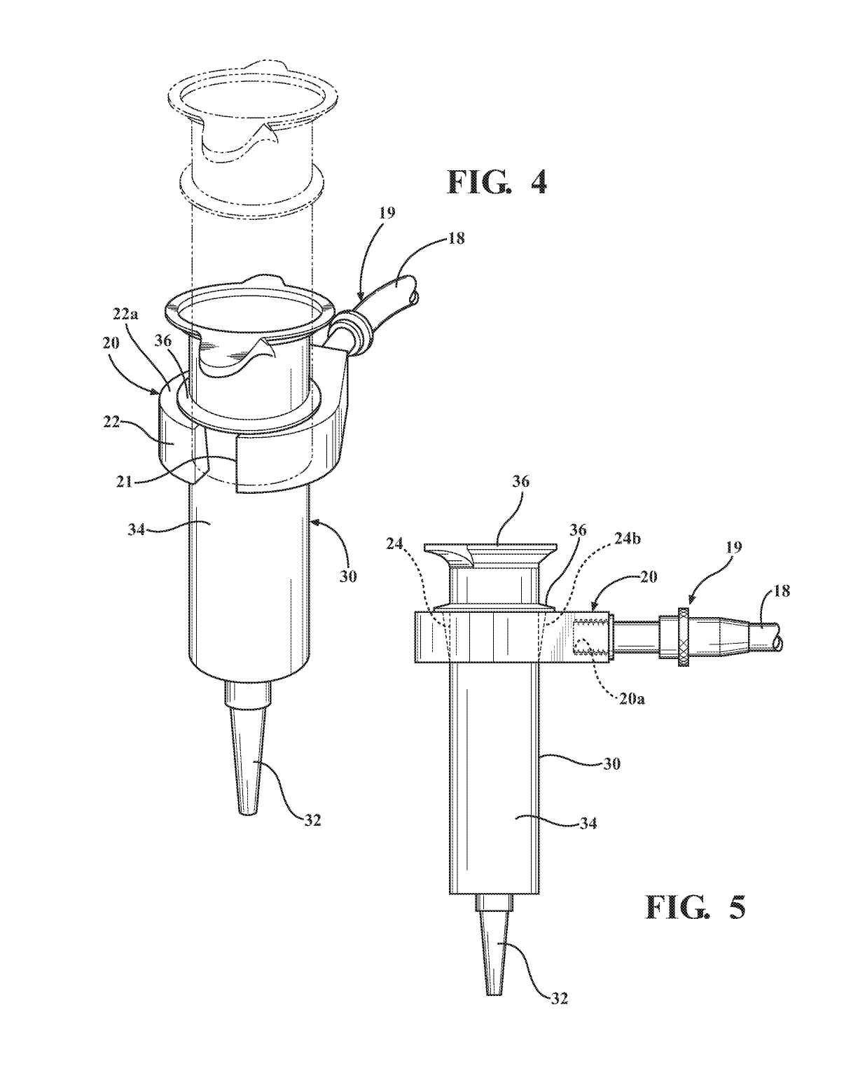

Referring next especially to FIGS. 4 and 5, the upper end of flex arm 18 terminates in a syringe receiver 20, in the illustrated example in the form of a rigid, non-expanding collar machined or cast from aluminum, or molded from a substantially non-flexing polymer, by way of non-limiting example. Receiver 20 may be connected to the end of flex arm 18 with a threaded connection 19 or other connection similar to that used to connect the lower end of the flex arm to post 16 and/or platform 14, engaging suitable mating structure such as a threaded bore in the rear of the collar. Receiver 20 includes a sidewall 22 and a bore 24, the bore sized to receive the body of a syringe 30 in an upside-down, drop-in manner, the bore further adapted to secure the syringe axially in place after the tip 32 and part of barrel 34 are extending from the bottom of the receiver. In one form, the bore 24 is cylindrical to match the most common syringe barrel geometry, and slightly larger than the diameter of the barrel 32 of the syringe for which it is adapted, such that the upper edge 22a of sidewall 22 engages a flange or shoulder 36 on the barrel to stop further descent through the receiver. In another form, the bore 24 is tapered, as shown in hidden lines at 24b in FIG. 5, wider at the top than the bottom, to help guide the syringe as it is being dropped into the collar.

While post 16, flex arm 18, and receiver collar 20 are illustrated as separate pieces which can be assembled and disassembled as needed, it would be possible to permanently connect them in various combinations.

Receiver collar 20 may optionally be split at a front part 21 of the sidewall 22, as best shown in FIG. 4, in order to provide a continuous view over the length of the syringe, either of the syringe contents or of graduation markings along the barrel of the syringe, and to provide a horizontal opening for a feeding tube to allow a syringe to be assembled to the feeding tube prior to inserting the syringe in the collar. Also, as shown in the drawings, the height of receiver collar 20 (the height of sidewall 22) is preferably substantially less than the height of the syringe.

Placing and securing the syringe 30 in receiver 20 is done by simply dropping the syringe vertically into the receiver, as represented by phantom lines in FIG. 4. This is accordingly a one-handed operation, a factor of convenience that can be especially important to busy caregivers or partially disabled patients.

FIG. 1 illustrates a caregiver 40 using syringe holder 10 to feed an infant “patient” 50. Holder 10 has been adjusted so that the syringe receiver 20 is at an optimum position and height for the caregiver's access and supervision, for the infant's comfort, and for efficient gravity feed of supplement from the syringe 30 through tube 38. Once gravity feeding begins, for example by releasing a clamp (not shown) on the tube 38, the caregiver has both hands free to care for and entertain infant 50. The continuous flexibility of arm 18 and the height adjustability of post 16 allow users a virtually limitless number of positions: sofa, floor, chair, bed or crib, etc.

FIG. 6 shows infant patient 50 receiving a tube feeding from a syringe 30 held by holder 10, without the need for immediate supervision. For example, a sleeping infant or patient may be fed while a caregiver is across the room, or in another room. Alternately, assuming the patient 50 is an able adult, once the tube feeding commences the patient is free to use his or her hands to read, operate a computer, or engage in other activities during the feeding.

FIG. 6 also shows a modified double receiver 120 on the end of flex arm 18, with two collar portions for holding two syringes 30 at a time. This may be useful for feeding multiple patients 50 (e.g., infant twins), or for holding an extra syringe of supplement handy for a quick second round of feeding.

The vertical split or opening 21 in sidewall 22 shown in FIGS. 1-6 above is shown centered on collar 20, and splits the sidewall 22 into two essentially identical halves. FIG. 7 shows a modified receiver collar 120, similar to receiver collar 20 above but with an off-center vertical split or opening 121 in sidewall 122, such that one sidewall portion 122a is longer than opposing sidewall portion 122b and terminates on the same side of the collar relative to a center axis X as the shorter portion 122b. The off-center split 121 allows a caregiver or user sitting at an angle or to the side of the holder apparatus 10 to see the syringe markings or contents more easily; allows for easier horizontal insertion of the feeding tube 30 into the collar's bore 24 if the feeding tube has already been connected to the syringe; and, given the adjustability of the flex arm 18 and/or a rotational coupling 119 between collar 120 and the flex arm, allows collar 120 to be used as a secure hook for feeding tubes, bags, or other items convenient for a user or caregiver to temporarily hang near the feeding location.

Referring to FIGS. 7 and 8, one or both of the connections 119 between the upper and lower ends of the flex arm 18 and collar 120 may be modified from threaded connections to quick-connect push fitting of known type. Quick-connect fitting 119 may comprise, for example, a male cylindrical stud 220 extending from the rear face of collar 120, either integrally or removably secured to the collar, with an annular groove 220a adapted to receive one or more spring-loaded balls or other detents 321 of known type situated internally in a mating female socket 320 on the end of the flex arm 19. An axially moveable release/locking sleeve 320a of known type may be used to selectively disengage or reduce the force of the internal detents in socket 320 to make the initial push-fit connection easier.

Quick-connect fitting 119 allows collar 120 to rotate on the end of the flex arm, so that the angular position of a syringe in the collar may be adjusted to some degree. In the illustrated example of FIGS. 7 and 8, quick-connect stud 220 includes a number of detent serrations or teeth 220b in groove 220a to provide a positively registered click, either by feel or sound, to the person adjusting the rotational angle of the collar, and to help lock the collar temporarily in a rotated position when supporting weight. The lower end of flex arm 18 may also be provided with a rotational quick-connect fitting 119, for example with male stud 220 on the tripod hub or post at 16a and the female socket 320 on the lower end of the flex arm.

FIG. 9 shows modified hook collar 120 rotated temporarily to a vertical position in which the “lower” or longer sidewall portion 122a can function as a hook, holding for example a feeding tube 30 up off the ground or table while the user is busy with some other task. Other uses for collar 120 as a temporary hook are possible and will be apparent to those who administer tube feedings.

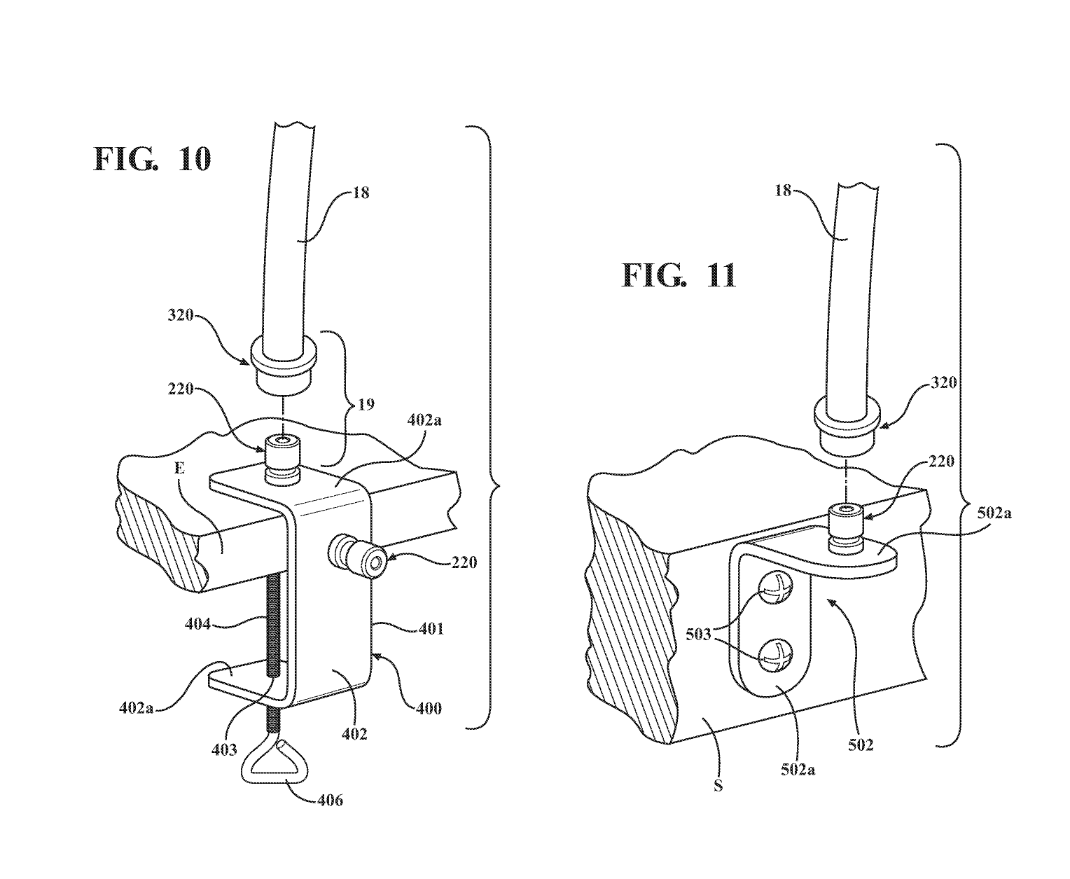

FIG. 10 shows a mounting clamp 400 designed to mount a flex arm 18 and one of collars 20 or 120 to the vertical or horizontal edge E of a table, chair, or similar structure (bench, shelf, etc.). In the illustrated example, clamp 400 includes a generally U-shaped yoke 401 with a main body 402 joining generally perpendicular yoke arms 402a and a clamping screw 404 threaded through a bore 403 in one of the yoke arms 402a. Clamping screw 404 is operated with a clamping knob 406 to turn and advance a pressure pad or disc (not shown, but of known type) on the end of screw 406 toward the inner face of the opposite yoke arm 402a to clamp edge E therebetween. Clamp 400 has one or more flex arm connections such as a male threaded post as in FIGS. 1-6 or a quick-connect stud 220 described above in FIGS. 7-9 mounted on one or more outer faces of the yoke 402, for example at right angles to one another in order to provide multiple mounting angles for the appropriate mating connector such as socket 320 on the lower end of the flex arm. Yoke 402 may be provided with soft pads on the inner faces of the yoke 401 opposite the connectors 220, the pads made from rubber, foam, plastic, etc. to protect edge E.

FIG. 11 shows a wall-mounting bracket 500 designed to mount a flex arm 18 via one of its lower end connections such as 320 to a flat furniture or wall surface S, whether vertical or horizontal. Illustrated example bracket 500 includes a generally L-shaped yoke 502 with arms 502a, one of which includes mounting holes for screws, bolts, or other mechanical connectors 503 used to secure the bracket to surface S. Similar to clamp 400 in FIG. 10, bracket 500 includes a male flex arm connection such as a male connection 220 described above mounted on one of the outer faces of the yoke, preferably the outer face of the arm 502a opposite the arm with screw holes 503.

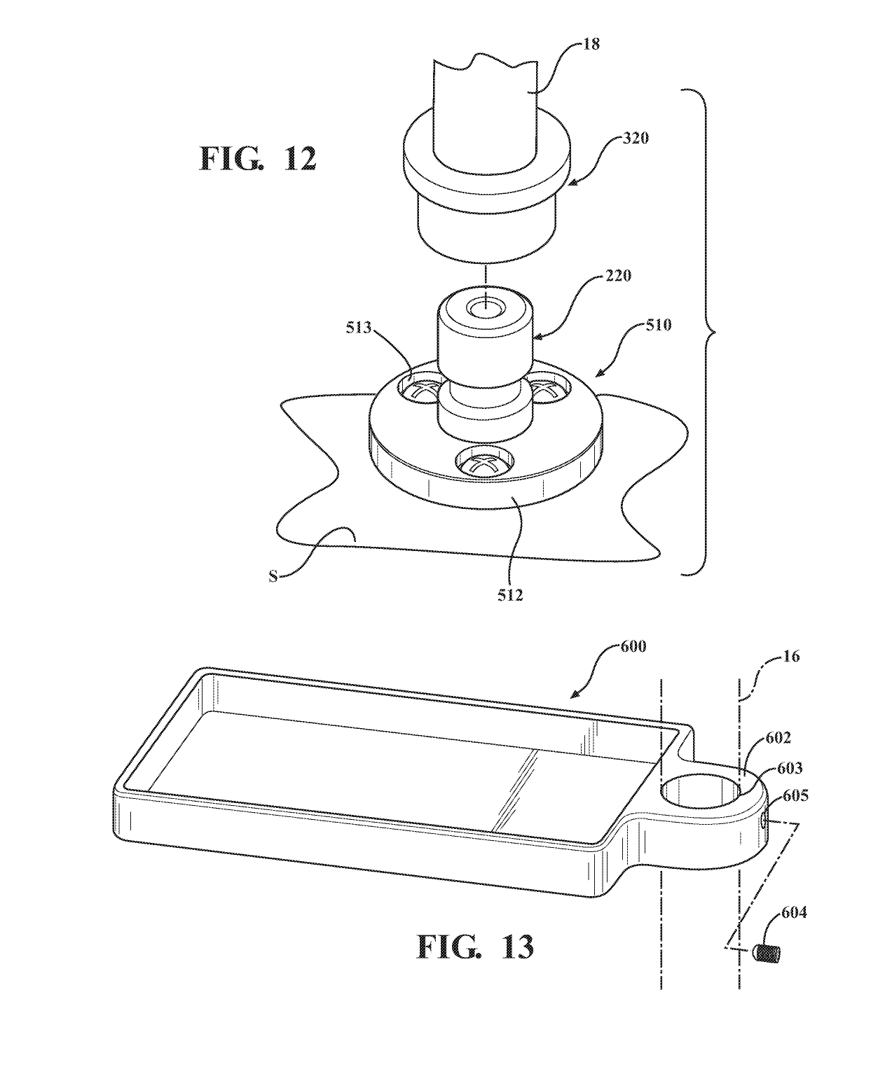

FIG. 12 shows a wall-mounting bracket 510 also designed to mount a flex arm 18 via a lower connector such as 320 to a flat surface S, whether vertical or horizontal. Illustrated example bracket 510 includes a generally flat base 512 with screw or similar connector mounting holes 513 used to secure the bracket to surface S. Similar to clamp 400 in FIG. 10, bracket 500 includes a male flex arm connection such as 220 described above mounted on one of the outer faces of the yoke, preferably the outer face opposite the arm 502a with screw holes 503.

FIG. 13 shows an accessory tray 600 configured to be mounted on the vertical post 16 of the tripod base 12 of FIGS. 1 and 2. Tray 600 in the illustrated example includes a post-mounting ear 602 with a bore 603 sized to fit closely over post 16, either to slide all the way down to the bottom of the post against the upper end of the tripod base at hub or platform 14, or by virtue of a set screw 604 inserted through a threaded hole 605 in ear 602 to be locked in place at different heights on post 16.

It will finally be understood that the disclosed embodiments represent presently preferred examples of how to make and use the invention, but are intended to enable rather than limit the invention. Variations and modifications of the illustrated examples in the foregoing written specification and drawings may be possible without departing from the scope of the invention. It should further be understood that to the extent the term “invention” is used in the written specification, it is not to be construed as a limiting term as to number of claimed or disclosed inventions or discoveries or the scope of any such invention or discovery, but as a term which has long been conveniently and widely used to describe new and useful improvements in science and the useful arts. The scope of the invention supported by the above disclosure should accordingly be construed within the scope of what it teaches and suggests to those skilled in the art, and within the scope of any claims that the above disclosure supports in this application or in any other application claiming priority to this application.

1. A tube feeding syringe holder for holding an inverted syringe, comprising:
a base;
a flex support arm connected to and extending from the base, the flex support arm being continuously bendable over its length to an essentially infinite number of self-sustaining positions within an arc or volume defined within the length of the flex support arm; and,
an axial syringe-receiving collar on the end of the flex support arm, the collar comprising a horizontal rigid non-expanding ring member with a fixed-diameter vertical throughbore, the collar movable on the flex support arm to an essentially infinite number of horizontal feeding positions relative to the tripod base within the arc or volume defined within the length of the flex support arm.

2. The tube feeding syringe holder of claim 1, further including an inverted syringe inserted axially into the vertical throughbore through an upper end of the throughbore and positively stopped and held in the collar by interference between a portion of the syringe and the collar.

3. The tube feeding syringe holder of claim 1, wherein the collar's vertical throughbore has a narrowing taper from the upper end to its lower end.

4. The tube feeding syringe holder of claim 1, wherein base comprises a freestanding portable tripod comprising three or more legs.

5. The tube feeding syringe holder of claim 4, wherein the flex support arm is secured at a lower end to a height-adjustable post mounted on the freestanding portable tripod.

6. The tube feeding syringe holder of claim 1, wherein the collar comprises a discontinuous sidewall defining the throughbore, the sidewall split by a vertical opening communicating with the throughbore.

7. The tube feeding syringe holder of claim 6, wherein the vertical opening is centered on the sidewall such that the sidewall is split into two portions of substantially equal length.

8. The tube feeding syringe holder of claim 6, wherein the vertical opening is offset on the sidewall such that the sidewall is split into a longer hook portion terminating on a first side of the collar and a shorter sidewall portion terminating on the first side of the collar.

9. The tube feeding syringe holder of claim 1, wherein the collar is rotatably adjustable on the flex support arm.

10. The tube feeding syringe holder of claim 6, wherein the collar is rotatably adjustable on the flex support arm.

11. The tube feeding syringe holder of claim 8, wherein the collar is rotatably adjustable on the flex support arm, and further wherein the collar is rotatable to a substantially vertical position in which the longer hook portion is located below the shorter sidewall portion to function as a hook.

12. The tube feeding syringe holder of claim 1, wherein the base comprises a generally U-shaped clamp comprising a yoke body joining two spaced arms, and a clamp screw threadably engaging one of the spaced arms for advancement of a pressure pad on the end of the clamp screw toward an inner surface of the other of the spaced arms, and further comprising a connection fitting on an exterior surface of the clamp mating with a lower end of the flex arm to mount the flex arm thereto.

13. The tube feeding syringe holder of claim 1, wherein the base comprises a mounting portion with a generally flat inner surface configured to be mechanically secured against a flat support surface, and further comprising a connection fitting on an exterior surface of the base mating with a lower end of the flex arm to mount the flex arm thereto.

14. The tube feeding syringe holder of claim 5, further comprising a horizontal tray configured to slidingly fit over the post and to be secured to the post in a desired position.
